(19) United States
(12) Patent Application Publication
(10) Pub. No.: US 2024/0066221 A1
(43) Pub. Date: Feb. 29, 2024

(54) CONNECTED DEVICE TO MEASURE INJECTED INSULIN

(71) Applicant: CeQur SA, Horw (CH)

(72) Inventors: Sivaramakrishnan Krishnamoorthy, Lexington, SC (US); Bradley Paddock, Greenville, SC (US)

(21) Appl. No.: 18/455,671

(22) Filed: Aug. 25, 2023

Related U.S. Application Data
(60) Provisional application No. 63/401,500, filed on Aug. 26, 2022.

(51) Int. Cl. A61M 5/168

(52) U.S. Cl. CPC A61M 5/1684

(57) ABSTRACT

A device for monitoring bolus doses of a medicament administered by a drug delivery device, such as a wearable infusion device, includes a housing and a sensor disposed therein, where the sensor detects a signal generated by the wearable infusion device upon administration of a dose of a medicament. The device may also be provided as a system with the drug delivery device and a user device, where the user device gathers data regarding use of the drug delivery device and doses of medicament administered.

### CROSS-REFERENCE TO RELATED APPLICATIONS

This application claims priority to and the benefit of U.S. Provisional Application No. 63/401,500, filed on Aug. 26, 2022, which is hereby incorporated by reference herein in its entirety.

### FIELD OF THE DISCLOSURE

The disclosure generally relates to monitoring dosing of a medicament and more particularly relates to systems and methods for monitoring bolus doses of a medicament administered by a pump device, such as a wearable patch pump.

### BACKGROUND

Insulin pumps may provide a user with constant basal delivery and/or bolus dosing of medicament (e.g., insulin). Bolus dosing can be activated by the user at any time, but bolus insulin dosing is typically initiated during mealtime. Certain existing bolus dosing systems and methods that rely on self-administration by the user may not track the number of insulin doses that have been self-administered. For example, in some devices, the doses administered correspond to the number of times a dosing button on the device has been pressed, and the user must count and remember the number of times the dosing button has been pressed. If the patient fails to accurately count or remember the number of times the button has been pressed, there is a risk of hyperglycemia (i.e., underdosing of insulin) or hypoglycemia (i.e., overdosing of insulin). Accordingly, improved devices and systems for tracking administration of insulin, particularly self-administered bolus doses, would be beneficial.

### SUMMARY

Devices and methods for monitoring and tracking bolus doses of a medicament administered by a drug delivery device are provided.

In one aspect, the device is configured for use with a wearable infusion device. In some instances, the device includes a housing and a sensor disposed within the housing. The sensor may be configured to detect and track a signal generated by the wearable infusion device upon administration of a dose of medicament. In some embodiments, the housing includes a first end and a second end, and the sensor may be disposed within the first end of the housing. The first end and/or the second end of the housing may be configured to facilitate releasable attachment of the device to the wearable infusion device. In some embodiments, the first end and the second end are connected by at least one flexible arm. For example, the at least one flexible arm may include two flexible arms. In some instances, a window (or void) may be formed between the flexible arms and the first and second ends of the housing. In some embodiments, the sensor is an accelerometer. In this manner, depression of at least one actuator button of the wearable infusion device may generate a signal detectable by the accelerometer. In some embodiments, the medicament is insulin. In some embodiments, the device is configured to be wirelessly connected to a user device, such as a computer and/or a mobile user device, such as a smartphone or the like. In some embodiments, the device also includes at least one attachment rail configured to facilitate releasable attachment of the device to the wearable infusion device.

In another aspect, a companion device for use with a wearable insulin pump is provided. The companion device may include a housing and an electronics assembly disposed within the housing. The companion device may be configured to be releasably attached to the wearable insulin pump. In some instances, the electronics assembly includes a sensor, a controller, and/or a battery. The sensor may be configured to detect a signal generated by the wearable insulin pump upon administration of a dose of insulin. In some embodiments, the controller and antenna are configured to track and wirelessly transmit the signal detected by the sensor to a user device and/or a wireless network. In some other embodiments, the signal is transmitted to an application installed on the user device, where the application displays a number of doses administered by the wearable insulin pump. In some embodiments, the housing includes a first end and a second end. In certain embodiments, the first end includes the electronics assembly, and the first and/or second end may be configured to facilitate releasable attachment of the companion device to the insulin pump. In some embodiments, the sensor is an accelerometer. The insulin pump also may include at least one dosing button, where the accelerometer is configured to detect a sound or vibration caused by depression of the at least one dosing button. In some embodiments, the companion device includes at least one attachment rail. In some embodiments, the at least one attachment rail extends at least partially along a length of the companion device. In some embodiments, the at least one attachment rail is configured to slide between a gap defined between an enclosure and a base of the insulin pump.

In yet another aspect, a system including an infusion device, a companion device, and a user device is provided. The companion device may include a housing and a sensor assembly disposed within the housing. The companion device may be configured to be removably coupled to the infusion device. The sensor assembly may be configured to collect data pertaining to the use of the infusion device and to transmit the data to the user device, such that the data is viewable by a user of the user device. In some embodiments, the system also includes an application installed on the user device. The application may be configured to display the data pertaining to the use of the infusion device. In some embodiments, the data corresponds to a number of medicament doses administered by the infusion device.

### BRIEF DESCRIPTION OF THE FIGURES

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular or plural terminology may be used interchangeably.

FIG. 1 is a front view of an infusion device, according to one or more embodiments of the present disclosure.

FIG. 2A is a front view of a companion device mechanically connected to the infusion device of FIG. 1, according to one or more embodiments of the present disclosure.

FIG. 2B is a perspective view of the companion device and infusion device shown in FIG. 2A, according to one or more embodiments of the present disclosure.

FIG. 3 is a back view of the companion device of FIGS. 2A-2B, separated from the infusion device, according to one or more embodiments of the present disclosure.

FIG. 4 is a partial cutaway view of the companion device of FIGS. 2A-2B and 3, according to one or more embodiments of the present disclosure.

FIGS. 5A-5C depicts a sequence of attaching the companion device to the infusion device, according to one or more embodiments of the present disclosure.

FIG. 6 is a diagram of an infusion system, according to one or more embodiments of the present disclosure.

FIG. 7 depicts a user interface of an application for use with an infusion device and/or a companion device, according to one or more embodiments of the present disclosure.

FIG. 8 is a perspective view of a companion device mechanically connected to the infusion device of FIG. 1, according to one or more embodiments of the present disclosure.

FIG. 9 is a front view of a companion device mechanically connected to the infusion device of FIG. 1, according to one or more embodiments of the present disclosure.

FIG. 10 is a front view of a companion device for the infusion device of FIG. 1, according to one or more embodiments of the present disclosure.

FIG. 11 is a front view of a companion device mechanically connected to the infusion device of FIG. 1, according to one or more embodiments of the present disclosure.

FIG. 12 is a front view of a companion device mechanically connected to the infusion device of FIG. 1, according to one or more embodiments of the present disclosure.

FIG. 13 is a perspective view of a companion device mechanically connected to the infusion device of FIG. 1, according to one or more embodiments of the present disclosure.

### DETAILED DESCRIPTION

The disclosure will be described more fully hereinafter with reference to the accompanying drawings, in which example embodiments of the disclosure are shown. This disclosure may, however, be embodied in many different forms and should not be construed as limited to the embodiments set forth herein.

Devices, systems, and methods have been developed for monitoring administration of a bolus dose of a drug delivery device, such as an insulin pump. In some embodiments, the monitoring device, referred to herein as a companion device, is an external device configured to fit over, or otherwise mechanically couple with, a wearable drug delivery device, which may include an insulin pump or patch and/or infusion device.

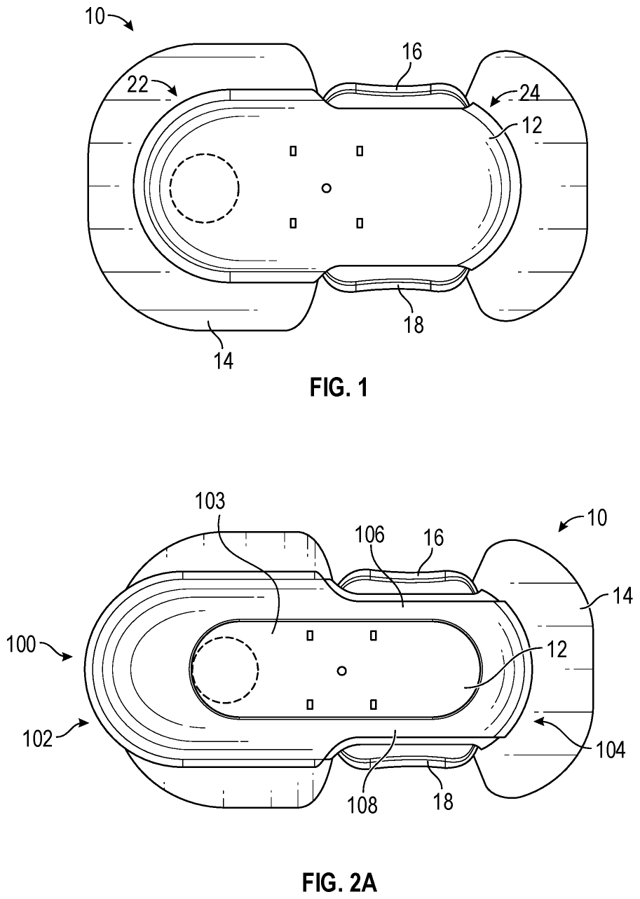

FIG. 1 depicts a wearable infusion device 10, such as an insulin pump or the like. In some embodiments, the infusion device 10 includes an enclosure 12, a base 14, a first actuator control button 16, and a second actuator control button 18. In some instances, the wearable infusion device 10 may correspond to the CEQUR SIMPLICITY® device sold by CeQur Corporation. In some embodiments, the enclosure 12 is formed of multiple device layers being brought together, as described in U.S. Pat. No. 8,128,596, which is incorporated by reference herein, in pertinent part. For example, each layer defines various components of the device, such as a reservoir, fluid conduits, pump chambers, and valve chambers. The device may include various other components and functionality.

In some embodiments, the base 14 includes an adhesive coating, e.g., a pressure sensitive adhesive tape as known in the art, to permit the device to be adhered to a patient's skin. In some embodiments, the adhesive coating is originally covered with a releasable cover that may be peeled off of the base 14 prior to patients adhering the device 10 to their skin. In some embodiments, the device 10 is adhered to the patient's skin prior to deployment of a cannula.

In some embodiments, the first actuator control button 16 and the second actuator control button 18 are placed on opposite sides of the infusion device 10 and directly across from each other. This renders more convenient the concurrent (or nearly concurrent) manual depression of the buttons when the patient wishes to self-administer a bolus dose of the liquid medicament contained within the infusion device 10. This arrangement also imposes substantially equal and opposite forces on the device during dosage delivery to prevent the device from being laterally displaced and possibly stripped from the patient. As will be further seen hereinafter, the substantially concurrent depression of the buttons is used to particular advantage.

Substantially concurrent depression of the first actuator control button 16 and the second actuator control button 18 may be particularly advantageous in preventing unintended medicament delivery. For example, given the wearable nature of the infusion device 10, the user may unintentionally disturb the infusion device 10, or more particularly, the first actuator control button 16 or the second actuator control button 18. Requiring that the first actuator control button 16 and the second actuator control button 18 be concurrently depressed for an intentional dosing advantageously militates against having an incidental or inadvertent contact with the infusion device 10 producing an unintentional dosing of the medicament.

In some embodiments, the actuator button 16 serves as a valve control which, when in a first position, establishes a first fluid path between the device reservoir and the device pump to support pump filling, and when in a second or depressed position, establishes a second fluid path between the device pump and the device outlet or cannula to permit dosage delivery to the patient. A linkage between the first control actuator button 16 and the second control actuator button 18 permits actuation of the device pump with the second actuator control button 18 only when the second fluid path has been established by the first actuator control button 16.

U.S. Pat. Nos. 8,226,607, 8,128,597, and 7,976,500 describe further embodiments and additional features of infusion devices operable with the companion device disclosed herein, each of which are incorporated herein by reference.

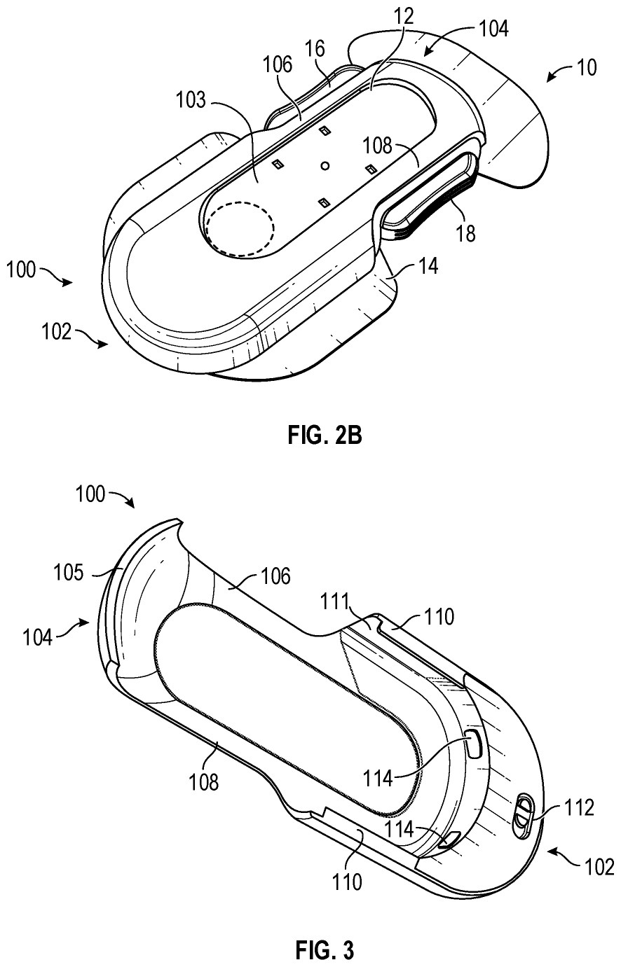

FIGS. 2A-4 depict a companion device 100, such as a connectable cover or a click counter, for use with wearable drug delivery devices, such as the infusion device 10 described with respect to FIG. 1. In certain embodiments, the infusion device 10 may be disposable, while the companion device 100 may be reusable. That is, the companion device 100 may be releasably attached to and detached from multiple infusion devices. For example, after each use, the companion device 100 may be removed from the infusion device prior to it being discarded, and then the companion device 100 may be reattached to a new infusion device for continued use thereof. When the companion device 100 is attached to a new infusion device 10, the connectivity between devices 100 and 10 may be reestablished, either manually or automatically.

In some embodiments, as shown in FIGS. 2A-2B, the companion device 100 has a first end 102, a second end 104, a first arm 106, and a second arm 108. In some instances, a window 103 (or void) is provided between the first end 102, the second end 104, the first arm 106, and the second arm 108. The window 103 may enable access to an outer surface of the infusion device 10 while the companion device 100 is attached thereto. The first end 102 may contain the operable components of the companion device 100, such as a battery, printed circuit board (PCB), microprocessor, wireless communication system and/or one or more sensors. The operable components may be disposed at any location about or within the companion device. The first end 102 and/or the second end 104 may be configured to secure the companion device 100 to the infusion device 10. The first arm 106 and the second arm 108 may extend between and connect the first end 102 and the second end 104 and function to maintain a secure connection of the companion device 100 to the infusion device 10 while also maintaining a relatively low profile so as to not become an impedance to the user's daily activity. In a preferred embodiment, the first arm 106 and the second arm 108 are disposed along the edges of the enclosure 12 adjacent to the first actuator control button 16 and the second actuator control button 18, respectively. Positioning the first arm 106 and second arm 108 along the medial portion of the infusion device 10 may prevent the companion device 100 from obstructing or interfering with the first actuator control button 16 and the second actuator control button 18. In this manner, the first arm 106 and second arm 108 may be shaped and/or include cutouts to prevent the companion device 100 from obstructing or interfering with the first actuator control button 16 and/or the second actuator control button 18. The first arm 106 and second arm 108 may be any suitable shape or configuration. In some embodiments, the first end 102 and/or the second end 104 of the companion device 100 follow the contours of the infusion device 10 for a better fit.

In some embodiments, the first arm 106 and second arm 108 may be flexible. In this manner, the first arm 106 and second arm 108 may flex when the companion device 100 is slid onto the infusion device 10. For example, the first arm 106 and second arm 108 may flex (elastically deform) outwardly away from the infusion device 10 as the companion device 100 is slid onto the infusion device 10 to enable the second end 104 to slide across the infusion device 10 and be positioned about the second end 24 of the infusion device 10. That is, the first arm 106 and the second arm 108 may flex outwardly away from the infusion device 10 to maintain the second end 104 of the companion device 100 in an outward position to facilitate attachment of the companion device 100 to the infusion device 10.

For example, to attach the companion device 100 to the infusion device 10, the first end 102 of the companion device 100 may be substantially aligned with (or slightly offset from) the first end 22 of the infusion device 10 and second end 104 of the companion device 100 may be substantially aligned with (or slightly offset from) the second end 24 of the infusion device 10 so that the companion device 100 can slide along the length thereof until the first end 102 of the companion device 100 abuts the first end 22 of the infusion device 10. For example, with reference to FIG. 2, the first companion device 100 may be offset to the left of the first end 22 of the infusion device 10, and slides to the right to become substantially aligned with and attached to the infusion device 10. Any tension in the first arm 106 and the second arm 108 from the outward flexing thereof may then be released to secure the second end 104 of the companion device 100 to the second end 24 of the infusion device. In some embodiments, the second end 104 of the companion device 100 includes a lip 105 that is also flexed outward during attachment, which then “snaps” onto the infusion device 10 to securely attach the companion device 100 thereto. The lip 105 may help to prevent inadvertent or unintended removal of the companion device 100 from the infusion device 10. For example, the lip 105 may prevent the companion device 100 from being pushed or slid of the infusion device 10 in the direction of the first end 22. In some embodiments, the bottom edge of the lip 105 has a tab (not shown) to help to further secure the companion device 100 to the infusion device 10. For example, when the second end 104 of the companion device 100 “snaps” onto the infusion device 10, the tab may be positioned in the gap 20 defined between the enclosure 12 and the base 14 of the infusion device 10.

In some embodiments, the companion device 100 is constructed of a biocompatible plastic, such as a polycarbonate plastic, similar to the enclosure 12 of the infusion device. In other embodiments, the companion device 100 is constructed of a shock-absorbent flexible material, such as silicone or rubber. In some embodiments, the companion device 100 is manufactured by a process that includes injection molding, with the potential for overmolding. In some embodiments, a custom logo may be printed on the connected cover 100.

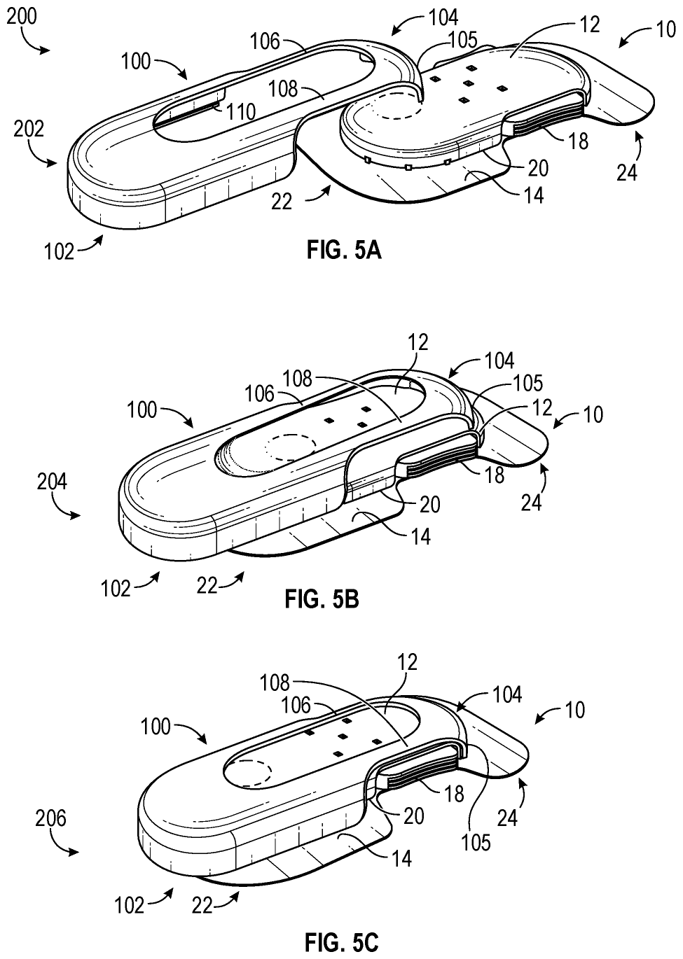

As shown in FIG. 3, the back side of the first end 102 of the companion device 100 may include attachment rails 110, an ON-OFF switch 112, and charging contacts 114. In some embodiments, the attachment rails 110 are integrally formed with the first end 102 of the companion device 100 to facilitate secure attachment of the companion device 100 to the infusion device 10, as described in greater detail with respect to FIG. 5. In some instances, the attachment rails 110 may be disposed on opposite sides of the first end 102 of the companion device 100. In this manner, each of the attachment rails 110 may form a channel 111 or the like in which at least a portion of the infusion device 10 may be slid into and secured in place. In some other instances, the attachment rails 110 may include a single attachment rail 110 extending at least partially around and inner portion of the first end 102 of the companion device 100 to form a single channel 111 in which at least a portion of the infusion device 10 may be slid into and secured in place. For example, the first end 22 of the infusion device 10 may be slid into the channel or channels 111 formed by the attachment rails 110. In some instances, the attachment rails 110 may be slid within the gap 20 between the enclosure 12 and the base 14 of the first end 22 of the infusion device 10 in order to attach the companion device 100 to the infusion device 10.

For example, when the first end 102 of the companion device 100 engages with the first end 22 of the infusion device 10 via attachment rails 110, the second end 104 of the companion device 100 may flex outward away from the infusion device 10 via flexing of the first arm 106 and second arm 108. Once the first end 102 of the companion device 100 is fully aligned with the first end 22 of the infusion device 10, the first arm 106 and the second arm 108 may force the second end 104 and the lip 105 downward around the second end of the infusion device 10. Securing the companion device 100 to the infusion device 10 in this manner may prevent unintended removal of the companion device. For example, the placement of the attachment rails 110 under at least a portion of the infusion device 10 may prevent the companion device 100 from being pulled out and away from the infusion device 10.

In some embodiments, the ON-OFF switch 112 permits a user to power on the companion device 100 when ready for use (e.g., for pairing to an infusion device 10 to a smart device) and power off the companion device following completion of use (e.g., to enable charging). In the ON position, the ON-OFF switch 112 may place the companion device 100 into a pairing mode where the companion device 100 may wirelessly connect to a user device and/or a network. The ON-OFF switch 112 may preferably be located on the back of the companion device 100 to prevent the user from unintentionally switching the ON-OFF switch 112 to the OFF position so that the companion device 100 remains operable at all times while the infusion device 10 is being worn. While the ON-OFF switch 112 is depicted and described herein as a switch, it would be understood that the ON-OFF switch 112 may also be a power button or the like, as would be understood by those skilled in the art.

In some embodiments, the companion device 100 includes one or more feedback indicators. In various embodiments, the feedback indicators may provide audible, visual, and/or haptic signals to the user/wearer of the companion device 100. For example, the feedback indicators may include one or more LED lights to provide visual feedback and/or vibrating indicators to provide tactile feedback. The feedback may pertain to the status of the ON/OFF switch. For example, the companion device 100 may be configured to display a flashing light or to vibrate when it is powered on, confirming to the user that it is operational. The companion device 100 may also be configured to display a flashing light or to vibrate to communicate to the user that the companion device 100 has a low battery or that the connection with a user device has been lost.

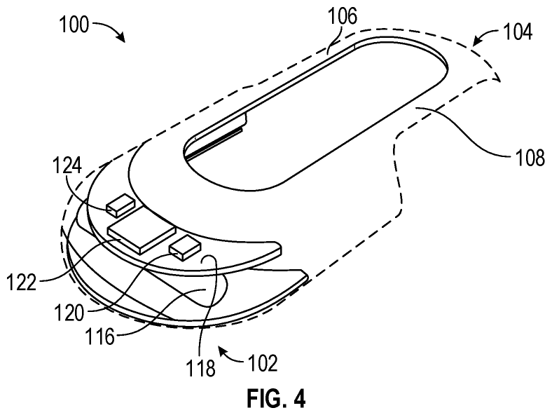

In some embodiments, the companion device 100 is powered by a rechargeable battery 116 disposed within the first end 102 of the companion device 100, as shown in FIG. 4. The battery 116 may be in electronic communication with the charging contacts 114 to charge the battery 116 without removing it from the companion device 100. In some preferred embodiments, the battery 116 powers the companion device 100 for at least 24 hours, such as up to 36 hours, up to 48 hours, or up to 72 hours. Any suitable battery may be used herein. In some embodiments, the battery 116 enters into a power saving mode (e.g., sleep mode) during periods of non-use to extend the life of the battery 116. It would be understood that, while charging contacts 114 are depicted, other charging means known in the art may be utilized. For example, the charging means may be a charging port, a charging dock, inductive charging, or the like.

In some embodiments, the battery 116 is an off-the-shelf battery. In other embodiments, the battery 116 is customized to fit with the contours of the infusion device 10 and/or the companion device 100. The battery 116 may be rechargeable, or the battery may be a single-use battery. In some other embodiments, other power sources known in the art may be used in addition to in place of the battery to power the companion device 100. In some instances, the battery may be in communication with one or more inductive coils for inductively charging the battery.

In some embodiments, an internal component of the ON/OFF switch 112 and the battery 116 are contained within the first end 102 of the companion device 100. As shown in FIG. 4, the first end 102 of the companion device 100 may also contain a PCB 118, a sensor 120, a microcontroller unit 122, and a wireless communication system 124, with an antenna such as Bluetooth or the like. Any suitable wireless protocols may be used herein. The companion device 100 may also have embedded wiring to connect the various electrical and/or power systems housed in the first end 102. Such electrical and/or power systems include, but are not limited to, leads for electronic connections, which can be imbedded within or printed on the first end 102 of the companion device 100.

The PCB 118, which may be a rigid or flexible PCB, and the microcontroller unit 122 may mechanically and electronically support the components of the companion device 100. Any suitable controller and/or processor and memory may be used herein. In some embodiments, the microcontroller unit 122 has software installed on a memory thereof. This software may, for example, receive data from the sensor and process the data using a signal processing algorithm to convert the raw data to a useful format. In some embodiments, the PCB 118 and/or microcontroller unit 122 are configured to be connected with a wireless module, such as Bluetooth or NFC module, or have an integrated Bluetooth or NFC module. The software installed on the microcontroller unit 122 may also push the raw and/or converted data to such a connected or integrated Bluetooth or NFC module.

In some other embodiments, the PCB 118 and/or the microcontroller unit may be connected with other electronic components, such as resistors, LEDs, feedback indicators, and/or additional circuitry contained within the companion device 100.

The sensor 120 may be configured to detect when the first actuator button 16 and the second actuator button 18 of the infusion device 10 are engaged. When the user (i.e., patient) wishes to receive a dose of medicament, the first actuator button 16 and second actuator button 18 are concurrently (or near concurrently) pressed to administer the medicament via the infusion device 10. The substantially concurrent pressing of the first actuator button 16 and the second actuator button 18 may generate a signal detectable by the sensor 120. In certain embodiments, the sensor 120 is configured to detect a unique profile (or signature) associated with concurrent pressing of the first actuator button 16 and the second actuator button 18. The signal may be indicative of the substantially concurrent pressing of the first actuator button 16 and the second actuator button 18, which is indicative of a dosing being administered by the infusion device 10. In a preferred embodiment, the sensor 120 is an accelerometer. In another embodiment, the sensor 120 may be a microphone or a switch. In another embodiment, the sensor may be a camera or the like. In this manner, depending on the sensor type, the unique profile (or signature) associated with concurrent pressing of the first actuator button 16 and the second actuator button 18 may include a movement profile, a vibration profile, a sound profile, or the like that is indicative of substantially concurrent pressing of the buttons and the dispensing of a dose. Other types of sensors may be used to sense any suitable unique profiles (or signatures).

In some embodiments, the companion device 100 has two or more sensors 120. A first sensor may be positioned proximate to the first actuator button 16 and the second actuator button 18, and a second (or subsequent) sensor may be positioned at a distance from the first actuator button 16 and second actuator button 18. Use of two or more sensors may prevent the companion device 100 from transmitting a signal indicating that a dose of medicament was administered when the first actuator button 16 and second actuator button 18 were not concurrently pressed. The second (or subsequent) sensor may be positioned at a predetermined distance from the first and second actuator buttons 16, 18 to achieve this purpose. For example, when a dose of medicament is administered (i.e., when the first actuator button 16 and the second actuator button 18 are concurrently pressed) the first sensor may detect a signal while the second sensor does not, indicating that a dose of medicament was administered. However, in the event of an unintended signal (e.g., a signal generated as a result of sound, movement, etc. unrelated to the concurrent pressing of the first actuator button 16 and second actuator button 18), the first and second (or subsequent) sensors may receive a signal, indicating that a dose of medicament was not administered.

In some embodiments, the first sensor is located in the companion device proximate to the first actuator button 16 and the second actuator button 18, and the second sensor is located at a second position in the companion device 100 at a distance from the first and second actuator buttons 16, 18. In some embodiments, the first sensor is located within the infusion device 10 on or proximate to the first and second actuator buttons 16, 18, and the second sensor is located in the companion device 100. In some embodiments, the first sensor is located in the companion device 100 proximate to the first and second actuator buttons 16, 18 and the second sensor is located in a user device, such as a mobile phone. In some embodiments, the first sensor is located in the companion device 100 proximate to the first and second actuator buttons 16, 18, and the second sensor is directly attached to the user's body (e.g., the second sensor is affixed to the base 14 of the infusion device 10. In some embodiments, the first sensor is located in a secondary device configured to be attached to the first and second actuator buttons 16, 18, and the second sensor is located in the companion device 100.

In some embodiments, the sensor 120 transmits the signal to a controller system, such as to the PCB 118 and/or microprocessor 122. In some instances, the PCB 118 and/or microprocessor 122 may process the signal from the sensor 120 and transmit the processed signal via the Bluetooth antenna 124 to a user device. The signal may indicate a number of administered medicament doses.

Electrical wires may connect the aforementioned components or the aforementioned components may all communicate wirelessly, as well as any additional components that one skilled in the art would recognize as comparable to those components disclosed herein.

FIGS. 5A-5C depicts a sequence of attaching the companion device to the infusion device, according to one or more embodiments of the present disclosure. As shown in FIG. 5A, the companion device 100 may be aligned with the infusion device 10 in preparation for attachment thereto. More specifically, the second end 104 of the companion device 100 may be aligned with the first end 22 of the infusion device 10, so that the user may then slide the companion device 100 towards the second end 24 of the infusion device 10. For example, the attachment rails 110 of the companion device 100 may be aligned with and slid into a gap 20 defined between the enclosure 12 and the base 14 of the infusion device 10, as shown in FIG. 5B, which may help to ensure proper and secure attachment between the companion device 100 and the infusion device 10. In some embodiments, the first arm 106 and the second arm 108 flex upward away from the infusion device 10 as the companion device 100 slides from the first end 22 to the second end 24 of the infusion device 10. Flexing of the first arm 106 and second arm 108 may distance the second end 104 of the companion device 100 from the infusion device 10 for ease of attachment. Once the first end 102 and second end 104 of the companion device 10 are aligned with the first end 22 and second end 24 of the infusion device 10, respectively, the second end 104 of the companion device 100 may be brought back towards and attached to the second end 24 of the infusion device 10, as shown in FIG. 5C. In some embodiments, a lip 105 on the second end 104 of the companion device 100 may “snap” onto the infusion device 10 to more securely attach the companion device 100. In some embodiments, the material of construction and the geometry of the first end 102 and the second end 104 are optimized for tactile feedback upon snapping.

In some embodiments, the companion device 100 is attached to the infusion device 10 after it has been affixed to the user's body. For example, the user may apply the infusion device 10 to a desired site on their body according to normal procedures, then align, slide, and snap the companion device 100 onto the already applied infusion device. In other embodiments, the companion device 100 is attached to the infusion device 10 prior to affixing the infusion device 10 to the user's body. The process for applying the infusion device 10 to the body while the companion device 100 is attached is substantially unchanged.

To remove the companion device 100, the first arm 106 and the second arm 108 may be flexed to lift second end 104 and/or the lip 105 of the companion device 100 outward away from the infusion device 10. The companion device 100 may then be slid towards the first end 22 of the infusion device 10 for removal. For example, the companion device 100 may slide towards the first end 22 of the infusion device 10 until the attachment rails 110 disengage with the infusion device 10, at which point the companion device 100 may be completely removed.

In some embodiments, the companion device 100 is attached to and/or removed from the infusion device 10 while it is attached to the user's body. In other embodiments, the companion device 100 is attached to and/or removed from the infusion device 10 when it is not affixed to the user's body.

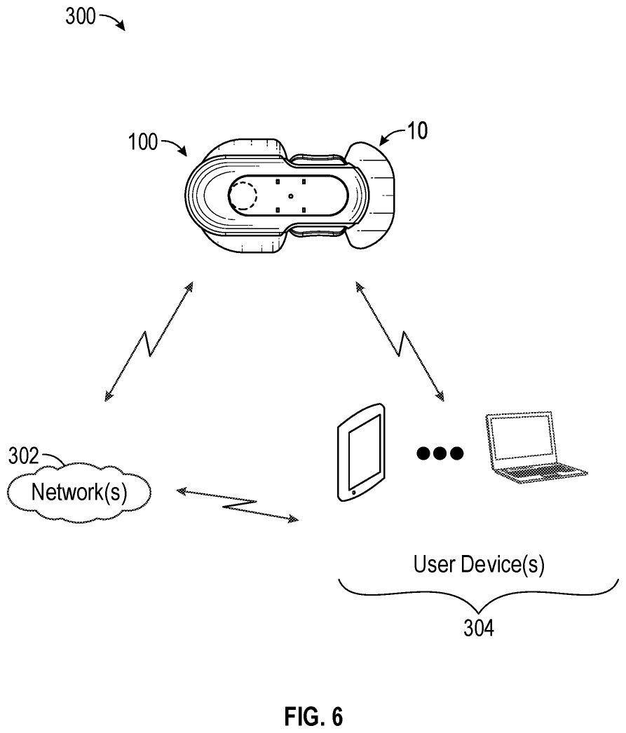

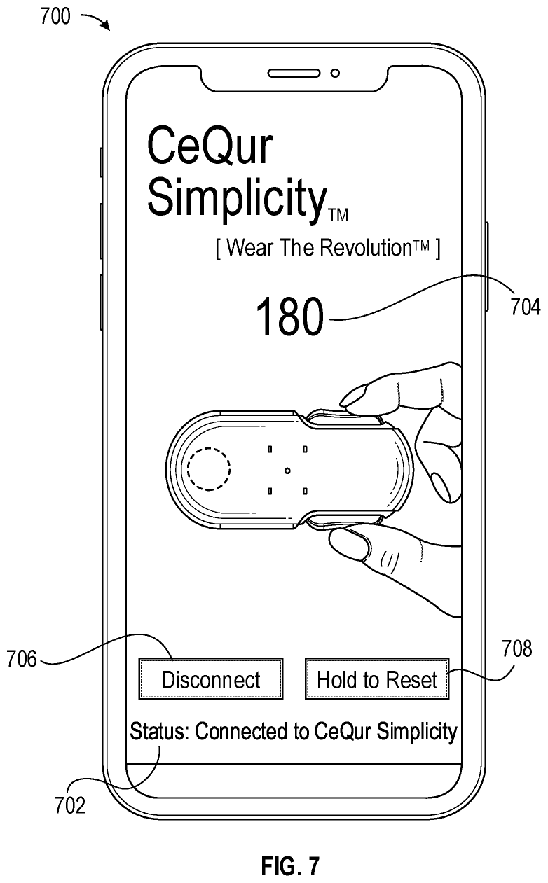

In some embodiments, the companion device 100 is configured to be connected to a user device and/or cloud network to facilitate dose monitoring. For example, as shown in FIG. 6, a system 300 for monitoring dose administration includes the companion device 100, a network (or plurality of networks) 302, and at least one user device 304, such as a smartphone, tablet, personal computer, server, and/or network. In some instances, a software installed on the microcontroller unit 122 of the companion device 100 receives, processes, and transmits sensor data regarding the number of medicament doses administered by the user. In some embodiments, this data is transmitted, via Bluetooth for example, to the user device 304 to be displayed on the user interface 700 of an application, as shown in FIG. 7. In other instances, raw unprocessed (or minimally processed) data may be transmitted.

In some embodiments, the user device 304 displays all information relevant to the operation of the infusion device 10 and/or companion device 100 on a user interface 700. For example, the user interface 700 may display information such as the status of 702 of the companion device 100, the battery level of the companion device 100, and the number of doses remaining 704. The user interface 700 may also include certain controls, such as a control for disconnecting 706 and/or resetting 708 the companion device 100.

In some embodiments, the companion device 100 is linked directly to a hospital information system, health data management system, diabetes management software, or remote patient monitoring system via a network 302. The network 302 may include any one, or a combination of networks, such as a local area network (LAN), a wide area network (WAN), a telephone network, a cellular network, a cable network, a wireless network, and/or private/public networks, such as the Internet. The network 302 may also support communication technologies, such as Bluetooth, cellular, near field communication (NFC), Wi-Fi, Wi-Fi direct, machine-to-machine communication, man-to-machine communication, and/or visible light communications.

In use, the system 300 enables accurate and reliable tracking of medicament doses delivered by the infusion device 10, and eliminates the need for users or their caretakers to manually track the administered doses. This may help with medication management by mitigating the risk that a user will administer too much or too little medicament, and/or that the user will waste or prematurely run out of medicament doses in the infusion device 10.

For example, as the companion device 100 counts the number of medicament doses administered by the infusion device 10, this count may be transmitted to the user device 304 and displayed on a user interface 700 of the application. In some embodiments, the application displays the number of remaining doses available. For example, the application initially displays the total number of doses available (e.g., 180), and as doses are administered the number of available doses will decrease in increments of two doses. In other embodiments, the application displays the number of doses administered. In some embodiments, the application is configured to display warnings, or provide the user with push notifications, indicating when, for example, the medicament supply in the infusion device 10 is low or when the user has delivered a predetermined maximum amount of medicament for a given time period. The user interface 700 may also display the time of administration to ensure patient compliance.

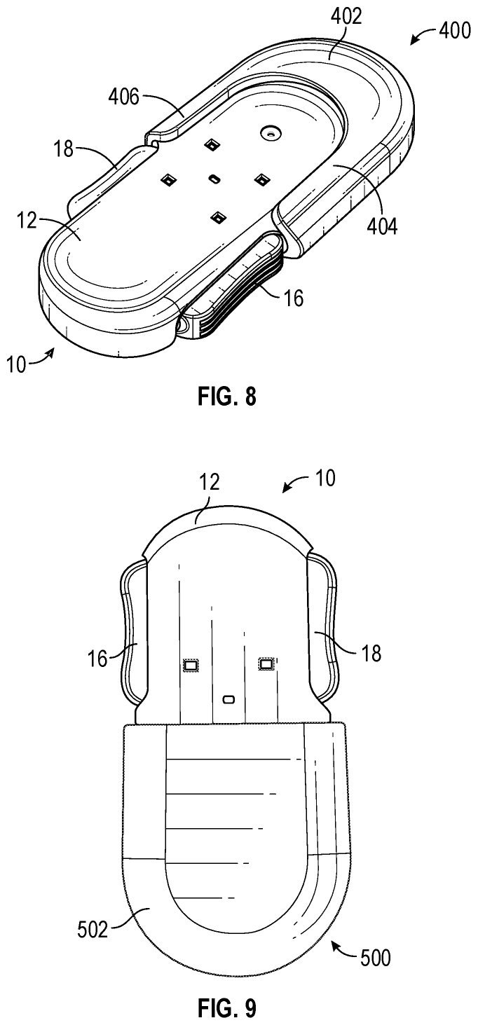

Referring now to FIGS. 8-13, alternative embodiments of a companion device for use with the infusion device 10 are described.

As shown in FIG. 8, the companion device 400 may be a U-shaped device resembling the first end 102 of the companion device 100. In some embodiments, the companion device 400 is similar to the companion device 100 described in FIGS. 1-7. For example, the companion device 400 may include a base portion 402 and a first arm 404 and a second arm 406 extending therefrom. The base portion 402 contains the operable components of the companion device 400, as described with respect to FIGS. 1-4.

In some embodiments, the companion device 400 may include attachment rails (not shown) to facilitate proper and secure attachment of the companion device 400 to the infusion device. The attachment rails may extend along at least a portion of companion device 400. That is, the base portion 402, the first arm 404, and/or the second arm 406 may have an attachment rail extending along at least a portion thereof. As described with respect to FIG. 3, the attachment rail may define a channel (not shown) for receiving the infusion device 10. For example, the companion device 400 may be aligned with and slid onto the first end 22 of the infusion device 10. When the companion device 400 is attached to the infusion device 10, the first arm 404 and the second arm 406 may abut, but not contact, the first actuator control button 16 and the second actuator control button 18.

In some embodiments, the companion device 500 includes a sleeve 502 configured to fit around at least a portion of the infusion device 10, as shown in FIG. 9. According to a preferred embodiment, the sleeve 502 is disposed around the first end 22 of the infusion device 10, abutting the first actuator button 16 and the second actuator button 18, so that the first actuator button 16 and second actuator button 18 remain unobstructed. In some embodiments, the sleeve 502 is formed of an elastically deformable material (e.g., a polyethylene, polypropylene, or other thermoplastic polymers) that slides onto the infusion device 10.

The companion device 500 may also include an adhesive pocket (not shown) that receives the infusion device 10 and companion device 500. In some embodiments, the adhesive pocket is formed of a flexible material that fits around the infusion device 10 and the companion device 500 to secure the companion device 500 to the infusion device 10 and/or to attach the infusion device 10 to the patient. For example, the adhesive pocket may be directly secured to the patient's skin when applying the infusion device 10. In some other embodiments, the companion device 500 has attachment rails (not shown), such as those described with respect to FIG. 3. The attachment rails may extend along at least a portion of the companion device 500 to facilitate attachment to the first end 22 of the infusion device 10. The attachment rails may define a channel for receiving and securing the infusion device 10 therein when the companion device 500 is attached.

In some embodiments, the companion device utilizes an external sensor to detect when medicament is administered by the infusion device 10.

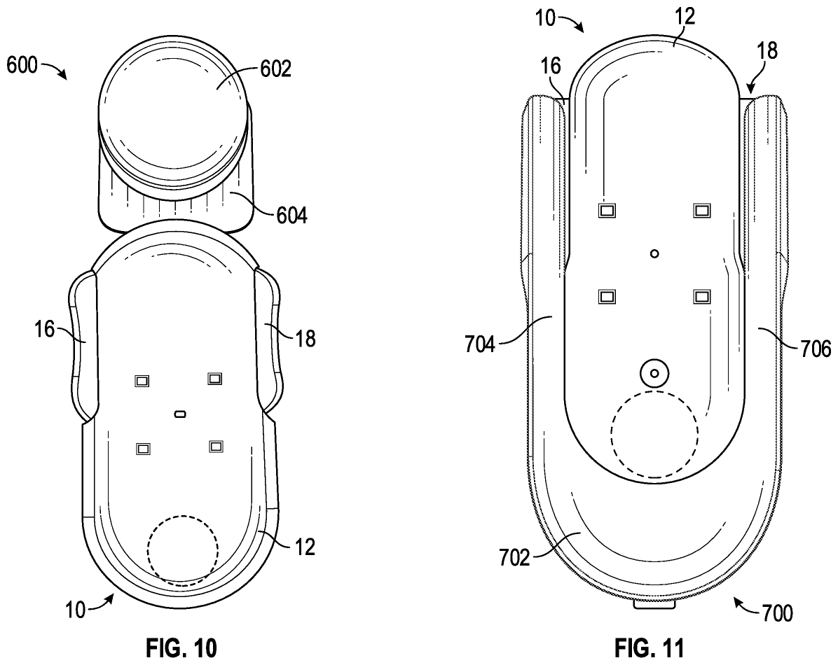

In some embodiments, the companion device 600 includes a microphone 602 for detecting when a dose is administered by the infusion device 10, as shown in FIG. 10. In some embodiments, the infusion device 10 may produce an audible sound when a dose is administered, this sound being detectable by the microphone. The companion device 600 may also include an adhesive patch 604 molded with a plastic knob (not shown), such that the microphone 602 is slidably attached to the adhesive patch via the molded plastic knob. In some preferred embodiments, the companion device 600 is placed as close to the infusion device 10 as possible to optimize sound detection and to limit interference. For example, as shown in FIG. 10, the companion device 600 may be placed directly next to the second end of the infusion device 10. In other embodiments, the companion device 600 is integral with the base 14 of the infusion device 10 to further simplify the user experience. For example, the companion device 600 may not include a separate adhesive patch, and is attached to the patient via the base 14 of the infusion device.

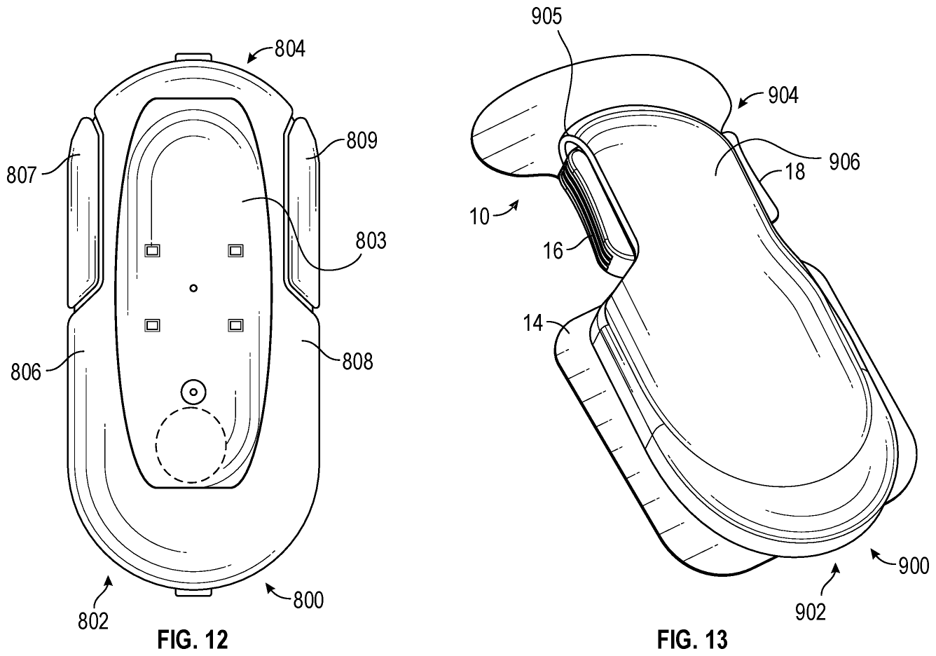

In some other embodiments, as shown in FIGS. 11-12, the companion device is configured to cover the first actuator button 16 and the second actuator button 18, and utilizes a force feedback sensor to detect when a dose has been administered.

As shown in FIG. 11, the companion device 700 is a U-shaped device having a base portion 702 housing the electronic components of the companion device 700, and a first arm 704 and a second arm 706 extending from the base portion 702 over the first actuator button 16 and second actuator button 18, respectively. The first arm 704 and second arm 706 may generally follow the contours of the first actuator button 16 and the second actuator button 18 include a sensor for detecting when the buttons 16 and 18 are engaged or pressed. That is, the first arm 704 and the second arm 706 may have a force and/or distance trigger threshold at which the sensor makes a determination that a dose should be counted. The first arm 704 and second arm 706 may be closely coupled to the infusion device 10 to ensure proper functioning of the sensor. However, the first arm 704 and the second arm 706 should not be so tightly coupled as to unintentionally depress the first actuator button 16 and/or the second actuator button 18.

In some embodiments, the first arm 704 and the second arm 706 are flexible so that the companion device 700 may be attached to the infusion device without compressing the first actuator button 16 and/or second actuator button 18. For example, the first arm 704 and second arm 706 may flex outwardly away from the infusion device 10 as the base portion 702 of the companion device 700 slides along the first end 22 of the infusion device 10. When the base portion 702 is secured to the infusion device 10, the first arm 704 and the second arm 706 may return to an at-rest state adjacent to the first actuator button 16 and the second actuator button 18.

In some other embodiments, the first arm 704 and the second arm 706 each include an elastic portion that facilitates coupling while also preventing the arms 704, 706 from compressing the actuator buttons 16, 18. The elastic portions may bias first arm 704 and the second arm 706 inwardly, but as the companion device 700 is coupled with the infusion device 10 the first arm 704 and the second arm 706 may be forced outwardly to receive the infusion device 10 therein. That is, the first arm 704 and second arm 706 may be forced outwardly as the base portion 702 of the companion device 700 slides along the first end of the infusion device 10. When the base portion 702 is secured to the infusion device 10, the first arm 704 and the second arm 706 may return to the at-rest state.

In some embodiments, the companion device 700 has attachment rails (not shown) for securing the companion device 700 to the infusion device 10, such as those described with respect to FIG. 3. The attachment rails may extend along at least a portion of the base portion 702, and/or along at least a portion of the first arm 704 and/or second arm 706. The attachment rails may also define a channel for receiving the infusion device 10 and securing the companion device 700 thereto.

In some embodiments, as shown in FIG. 12, the companion device 800 surrounds the entire perimeter of the infusion device 10. In some embodiments, the companion device 800 includes a first end 802 containing the electronic components of the companion device 800 and a second end 804 for securing the companion device 800 to the infusion device 10. The companion device 800 may also include a first arm 806 and second arm 808 connecting the first end 802 and the second end 804 of the companion device, defining a window 803 therebetween. The first arm 806 and the second arm 808 may also cover the first actuator button 16 and the second actuator button 18. In some embodiments, the first arm 806 and second arm 808 have a first button cover 807 and a second button cover 809, respectively, covering the first and second actuator buttons 16, 18.

The first and second button covers 807, 809 follow the contours of the first actuator button 16 and the second actuator button 18, and include a sensor, such as a switch, to determine when the first actuator button 16 and the second actuator button 18 are depressed. In some embodiments, the first button cover 807 and second button cover 809 are formed to prevent unintentional depression of the first actuator button 16 and the second actuator button 18. In some embodiments, the first button cover 807 and second button cover 809 are spring-loaded caps. In other embodiments, the first button cover 807 and second button cover 809 are co-molded elastomers.

In some embodiments, the companion device 800 snaps onto the infusion device 10 via at least one spring-loaded latch at either end, or at both ends, of the companion device. That is, one or both of the first end 802 and the second end 804 are flexible so that companion device 800 may be fitted over the infusion device 10. The first arm 806 and/or the second arm 808 may also be flexible for this same purpose. In other embodiments, the companion device 800 is separable into two pieces, each piece configured to slide onto the infusion device 10 at opposing ends and to fit together to secure the companion device 800 to the infusion device. For example, each piece of the companion device may have an attachment rail so that the companion device 800 may slide onto the infusion device 10, as described with respect to FIG. 3.

In further embodiments, as shown in FIG. 13, the companion device 900 covers the entire surface of the enclosure 12 of the infusion device 10, leaving the first actuator button 16 and the second actuator button 18 accessible. In some embodiments, the companion device 900 has a first end 902, a second end 904, and a flexible arm portion 906 extending therebetween. As described with respect to FIG. 3, the companion device 900 may have attachment rails (not shown) to facilitate attachment of the companion device 900 to the infusion device 10. For example, the first end 902 may have an attachment rail extending along at least a portion thereof, the attachment rail defining a channel into which the first end 22 of the infusion device 10 may be received. To attach the companion device 900 to the infusion device, the second end 904 of the companion device 900 may be aligned with and advanced along the infusion device 10 towards the second end 24. The flexible arm portion 906 and/or the second end 904 may be flexed outward away from the infusion device 10 to facilitate this attachment. When the first end 902 of the companion device 900 is aligned with the first end 22 of the infusion device 10, the second end 904 of the companion device may be brought inward toward the device and secured to the second end 24 of the infusion device. The companion device 904 may also have a lip 905 attached to the second end 904 to further secure this attachment.

In some embodiments, the infusion device 10 includes a magnet positioned between the first actuator button 16 and the second actuator button 18 that is configured to move when the first actuator button 16 and the second actuator button 18 are depressed. The companion device 900 has a corresponding magnet or sensor that is configured to detect movement of the actuator buttons within the infusion device 10. Companion device 900 may have the structure of and may attach to the infusion device 10 as described with respect to any of the previously described embodiments, as described in FIGS. 1-12.

While the disclosure has been described with reference to a number of exemplary embodiments, it would be understood by those skilled in the art that the disclosure is not limited to such embodiments. Rather, the disclosed embodiments can be modified to incorporate any number of variations, alterations, substitutions, or equivalent arrangements not described herein, but which are commensurate with the spirit and scope of the disclosure.

That which is claimed is:

1. A device for use with a wearable infusion device, the device comprising:
a housing configured for attachment to a wearable infusion device; and
a sensor disposed within the housing;
wherein the sensor is configured to detect a signal generated by the wearable infusion device upon administration of a dose of a medicament.

2. The device of claim 1, wherein the housing comprises a first end and a second end, wherein the sensor is disposed within the first end of the housing, and wherein the second end of the housing is configured to facilitate releasable attachment of the device to the wearable infusion device.

3. The device of claim 2, wherein the first end and the second end are connected by at least one flexible arm.

4. The device of claim 1, wherein the sensor is an accelerometer.

5. The device of claim 4, wherein depression of at least one actuator button of the wearable infusion device generates the signal, and wherein the signal is detectable by the accelerometer.

6. The device of claim 1, wherein the wearable infusion device is a wearable patch pump, and wherein the medicament is insulin.

7. The device of claim 1, wherein the device is configured to be wirelessly connected to a user device.

8. The device of claim 1, further comprising at least one attachment rail configured to facilitate releasable attachment of the device to the wearable infusion device.

9. A companion device for use with a wearable insulin pump, the companion device comprising:
a housing; and
an electronics assembly, disposed within the housing, comprising a sensor, a controller, and a battery,
wherein the companion device is configured to be releasably attached to the wearable insulin pump, and
wherein the sensor is configured to detect a signal generated by the wearable insulin pump upon administration of a dose of insulin.

10. The companion device of claim 9, wherein the controller is configured to wirelessly transmit the signal detected by the sensor to a user device and/or a wireless network.

11. The companion device of claim 10, wherein the signal is transmitted to an application installed on the user device, wherein the application is configured to display a number of doses of insulin administered by the wearable insulin pump.

12. The companion device of claim 9, wherein the housing comprises a first end configured to contain the electronics assembly therein and a second end configured to facilitate releasable attachment of the companion device to the insulin pump.

13. The companion device of claim 9, wherein the sensor is an accelerometer.

14. The companion device of claim 13, wherein the insulin pump comprises at least one dosing button, and wherein the accelerometer is configured to detect a sound or a vibration caused by actuation of the at least one dosing button.

15. The companion device of claim 9, further comprising at least one attachment rail configured to facilitate releasable attachment of the companion device to the insulin pump.

16. The companion device of claim 15, wherein the at least one attachment rail extends at least partially along a length of the companion device.

17. The companion device of claim 15, wherein the at least one attachment rail is configured to slide between a gap defined between an enclosure of the insulin pump and a base of the insulin pump.

18. A system comprising:
an infusion device;
a companion device comprising a housing and a sensor assembly disposed within the housing; and
wherein the companion device is configured to be removably coupled to the infusion device, and
wherein the sensor assembly is configured to detect data pertaining to the use of the infusion device and transmit the data to a user device, such that the data is viewable by a user of the user device.

19. The system of claim 18, further comprising an application installed on the user device, wherein the application is configured to display the data pertaining to the user of the infusion device.

20. The system of claim 18, wherein the data corresponds with a number of medicament doses administered by the infusion device.
